(19) United States
(12) Patent Application Publication
(10) Pub. No.: US 2016/0346416 A1
(43) Pub. Date: Dec. 1, 2016

(54) LOW RELATIVE HUMIDITY DECONTAMINATION SYSTEM

(71) Applicant: Mar Cor Purification, Minneapolis, MN (US)

(72) Inventors: Mason Schwartz, Elk River, MN (US); Jonathan K. Olson, Maple Grove, MN (US)

(21) Appl. No.: 15/161,498

(22) Filed: May 23, 2016

Related U.S. Application Data
(60) Provisional application No. 62/167,139, filed on May 27, 2015.

(51) Int. Cl. A61L 2/20; A61B 90/70

(52) U.S. Cl. CPC A61L 2/20; A61B 90/70

(57) ABSTRACT

A method of decontaminating a device comprising dispersing a decontamination fluid as a vapor into a decontamination chamber containing a device to be decontaminated, the decontamination fluid containing peracetic acid and water; controlling a dispersion rate of the decontamination fluid such that the environment within the decontamination chamber is below 45% relative humidity throughout a decontamination cycle defined from when the device is placed in the decontamination chamber to when the device is removed from the decontamination chamber, wherein the decontamination cycle achieves a sterility assurance level of at least 10 to the minus three.

### CROSS-REFERENCE TO RELATED APPLICATION

This application claims the benefit of priority to U.S. Provisional Application No. 62/167,139, filed May 27, 2015, which is herein incorporated by reference in its entirety.

### TECHNICAL FIELD

The present invention relates to decontamination of medical devices. More particularly, the present invention relates to a system for decontaminating a medical device in a sterilization chamber with an environment at least partially controlled by the components of the decontaminating substance.

### BACKGROUND

Medical equipment is often sterilized at high temperatures. Commonly, the equipment is sterilized in a steam autoclave under a combination of high temperature and pressure. While such sterilization methods are effective for more durable medical instruments, advanced medical instruments formed of rubber and plastic components with adhesives are delicate and unsuited to the high temperatures and pressures associated with a conventional steam autoclave.

Steam autoclaves have also been modified to operate under low pressure cycling programs to increase the rate of steam penetration into the medical devices or associated packages of medical devices undergoing sterilization. Steam sterilization using gravity, high pressure or pre-vacuum create an environment where rapid changes in temperature can take place. In particular, highly complex instruments which are often formed and assembled with very precise dimensions, close assembly tolerances, and sensitive optical components, such as endoscopes, may be destroyed or have their useful lives severely curtailed by harsh sterilization methods employing high temperatures, high or low pressures, and high levels of relative humidity.

Further, endoscopes in particular present problems in that such devices typically have numerous exterior crevices and interior lumens which can harbor microbes. The employment of a fast-acting yet gentler sterilization method is desirable for reprocessing sensitive instruments such as endoscopes. Other medical or dental instruments which comprise lumens, crevices, and the like are also in need of methods of cleaning and decontaminating which employ an effective reprocessing system which will minimize harm to sensitive components and materials.

### SUMMARY

A decontamination apparatus for decontaminating a device, comprising a decontamination chamber, an atomizing nozzle in fluid communication with a decontamination fluid containing water and peracetic acid, the atomizing nozzle configured to supply the decontamination fluid to the decontamination chamber as a vapor; and a control system configured to control a flow rate of the decontamination fluid into the decontamination chamber such that the relative humidity within the decontamination chamber is maintained between 0% and 45% throughout a complete chemical injection cycle, and wherein the control system is configured to control a decontamination cycle that includes the chemical injection cycle to achieve a sterility assurance level of at least 10 to the minus three (10⁻³).

A method of decontaminating a device comprising dispersing a decontamination fluid as a vapor into a decontamination chamber containing a device to be decontaminated, the decontamination fluid containing peracetic acid and water; controlling a dispersion rate of the decontamination fluid such that the environment within the decontamination chamber is below 45% relative humidity throughout a decontamination cycle defined from when the device is placed in the decontamination chamber to when the device is removed from the decontamination chamber, wherein the decontamination cycle achieves a sterility assurance level of at least 10 to the minus three (10⁻³).

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of an embodiment of a system for decontaminating a medical device.

FIG. 2 is a cutaway view of an atomizing nozzle, according to some embodiments.

FIG. 3 is a flow diagram of an exemplary process for decontaminating a medical device in the system of FIG. 1.

FIG. 4 is a graph of the relationship between pressure versus time during an example decontamination cycle in the system of FIG. 1, according to some embodiments.

FIG. 5 is a graph of the relationship between pressure and relative humidity versus time during an exemplary decontamination cycle in the system of FIG. 1, according to some embodiments.

### DETAILED DESCRIPTION

Certain devices, for example delicate medical instruments, may be unsuited for decontamination in an environment with a high relative humidity. For example, medical devices incorporating certain adhesives may be damaged by prolonged periods of time in an environment with a relative humidity over 50%. In another example, some enzyme based glucose testing requires a relative humidity low enough to allow the enzymes to survive the decontamination process. On the other hand, decontamination techniques are often more effective at a higher relative humidity. For this reason, there is a need for a decontamination technique that operates at a relative humidity low enough to handle delicate medical devices yet remains effective in reducing microbial activity. Relative humidity refers to the amount of water vapor in the atmosphere as a percentage of the amount of water vapor needed for saturation at the same temperature and pressure.

A decontamination process begins when a device is placed in the decontamination chamber, and ends when the device is removed from the decontamination chamber. In some embodiments, a decontamination process may include two or more decontamination cycles. In some embodiments, a vaporized decontaminating fluid containing peracetic acid is used to decontaminate a device, such as a medical device, while the relative humidity of the decontaminating environment is no greater than 45% during any portion of the decontamination cycle. In another embodiment, the relative humidity in the decontaminating environment is kept at or above 20% and below 45% during the decontamination cycle. In another embodiment, the relative humidity in the decontaminating environment is kept below 20% during the decontamination cycle.

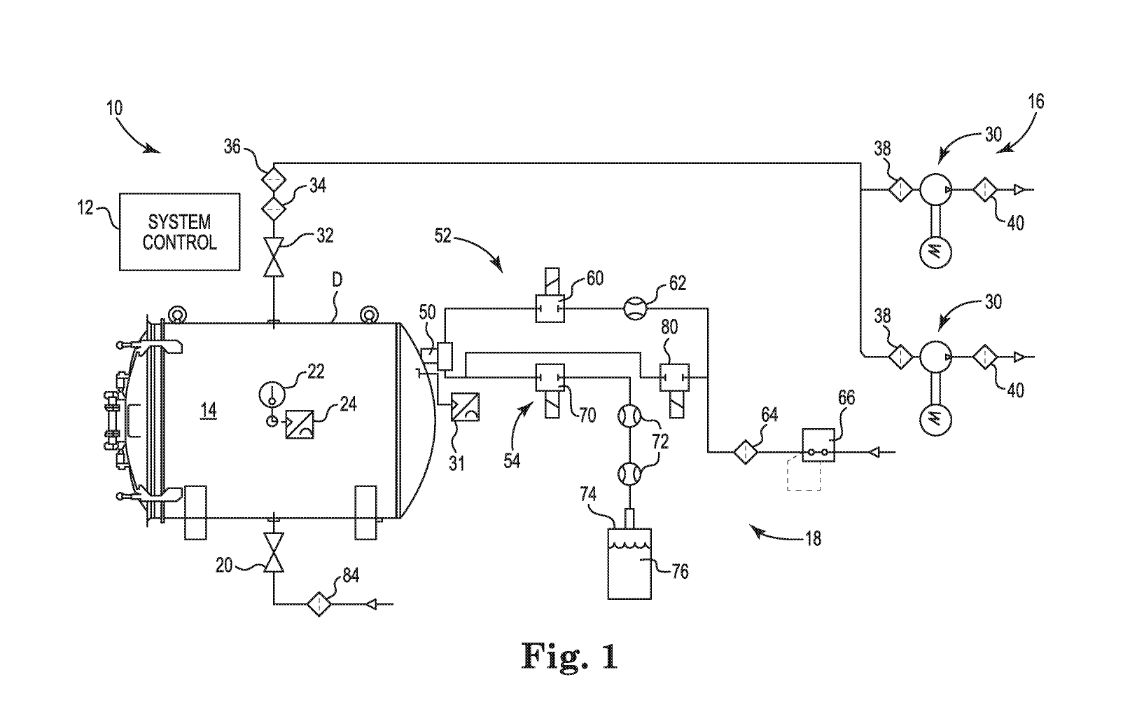

FIG. 1 is a schematic view of an embodiment of a system 10 for decontaminating a medical, dental, or other device. The system 10 includes a system controller 12, a decontamination chamber 14, a pressure control assembly 16, a chemical dispersion assembly 18, and a vent valve 20. The system controller 12 provides control signals to and/or receives control signals from components of the decontamination chamber 14, pressure control assembly 16, chemical dispersion assembly 18, and vent valve 20. In some embodiments, the system controller 12 is provided on the decontamination chamber 14 and is operatively connected to the components of the system 10. The system controller 12 may include an interface (e.g., on the decontamination chamber 14) that allows a user of the system 10 to provide inputs and receive information regarding operation of the system 10. For example, the system controller 12 may include an input device (e.g., a touch screen or keypad) that facilitates selection of decontamination cycle and/or chemical injection cycle parameters by the user.

The decontamination chamber 14 is an enclosure that is fluidly connected to the pressure control assembly 16, chemical dispersion assembly 18, and vent valve 20. The device to be decontaminated is placed into the decontamination chamber 14 by opening the door D and placing the device to be decontaminated on a rack or other supporting assembly in the interior of the decontamination chamber 14. When the door D is closed, the interior of the decontamination chamber 14 is sealed.

The decontamination chamber 14 may include a temperature sensor 22 and/or a relative humidity sensor 24 configured to monitor one or more environmental conditions in the decontamination chamber 14. In some embodiments, the temperature sensor 22 and/or the relative humidity sensor 24 may be operatively connected to the system controller 12. In some embodiments, as described herein, the system controller 12 may adjust or regulate one or more parameters, such as fluid flow rate and/or pressure, based on information received from the temperature sensor 22 and/or the relative humidity sensor 24. For example, in some embodiments, the system controller 12 may adjust or regulate fluid flow rate and/or pressure based on information received from the temperature sensor 22 to maintain the relative humidity within the decontamination chamber 14 between 0% and 45% through a complete chemical injection cycle and/or decontamination cycle. Additionally or alternatively, some embodiments, the system controller 12 may adjust or regulate fluid flow rate and/or pressure to achieve a sufficient sterility assurance level as described herein.

In some embodiments, the decontamination chamber 14 may be allowed to remain at the temperature of the surroundings throughout the decontamination cycle. For example, in some embodiments, the decontamination chamber 14 may be at ambient or room temperature throughout the decontamination cycle. In some embodiments, the decontamination chamber 14, may be maintained within a suitable temperature range throughout a decontamination cycle. For example, the decontamination chamber 14 may be maintained from about 1° C. to about 100° C., from about 2° C. to about 55° C., from about 3° C. to about 30° C., from about 4° C. to about 20° C., or any temperature within. In some embodiments, the decontamination chamber 14 may be maintained at 30° C. or below throughout the decontamination cycle. For example, in some embodiments, the decontamination chamber 14 may be maintained from about 4° C. to about 55° C. In some embodiments, the decontamination chamber 14 may be maintained from about 4° C. to about 18° C. In some embodiments, the decontamination chamber 14 may be maintained from about 18° C. to about 55° C. In some embodiments, the decontamination chamber 14 may be maintained from about 30° C. to about 55° C. throughout a decontamination cycle.

The pressure control assembly 16 may include one or more vacuum pumps 30 configured to evacuate the decontamination chamber 14 to produce a vacuum or partial vacuum in the decontamination chamber 14. In the embodiment shown, the pressure control assembly 16 includes two vacuum pumps 30. A vacuum control valve 32 may be disposed between the decontamination chamber 14 and the vacuum pumps 30 and may be configured to control fluid communication between the decontamination chamber 14 and the vacuum pumps 30. In an exemplary embodiment, the vacuum pumps 30 are configured to draw up to 300 liters per minute (L/min). A pressure sensor or transducer 31 may be operatively coupled to the decontamination chamber 14 to measure and monitor the pressure inside the decontamination chamber 14.

To destroy or neutralize decontaminating substances that are drawn from the decontamination chamber 14 during operation of the system 10, the pressure control assembly 16 may include one or more filters disposed between the vacuum control valve 32 and the vacuum pumps 30. In the embodiment shown, the pressure control assembly 16 includes filters 34, 36, 38 disposed between the vacuum control valve 32 and vacuum pumps 30. The type and number of filters employed in the pressure control assembly 16 may be a function of the type of decontaminating substance used in the system 10. For example, in some embodiments, the decontaminating substance includes hydrogen peroxide (H₂O₂), peracetic acid (PAA), and acetic acid. To neutralize this substance, the filter 34 may comprise potassium permanganate to remove the H₂O₂ and PAA, the filter 36 may comprise a particulate filter to remove particulate matter generated by the filter 34, and the filter 38 may comprise sodium bicarbonate to remove the acetic acid. In the embodiment shown, a filter 38 is associated with each vacuum pump 30. At the exit of the filters 38, the air drawn through the vacuum pumps 30 comprises oxygen (O₂) and carbon dioxide (CO₂). In some embodiments an exhaust filter 40 is connected to the outputs of each of the vacuum pumps 30.

The chemical dispersion assembly 18 may disperse a decontaminating substance in the decontamination chamber 14 during operation of the system 10. In some embodiments, the chemical dispersion assembly 18 may be fluidly connected to the device to be decontaminated. For example, a fluid connection may couple the chemical dispersion assembly 18 to the device being decontaminated. In some embodiments, the device to be decontaminated may be enclosed in a package or container. In some embodiments, the package or container may be impermeable by the decontaminating substance. In other embodiments, the decontaminating substance may be able to permeable through at least a portion of the packaging or container. For example, all or at least a portion of the packaging or the container may include Tyvek®, or other materials such as PET or Mylar®. In some embodiments, ethylene oxide needs a relative humidity greater than 50% to penetrate through packaging made of such material. In some embodiments, the packaging may be fluidly connected to the chemical dispersion assembly 18 and/or the device within the packaging may be fluidly connected to the chemical dispersion assembly 18. For example, a fluid connection may couple the packaging and/or the device within the packaging to the chemical dispersion assembly 18.

The chemical dispersion assembly 18 may include a nozzle 50 connected to an air flow subassembly 52 and a chemical flow subassembly 54. The air flow subassembly 52 may include an air flow control valve 60, an air flow meter 62, an air filter 64, and an air pressure regulator 66. The air pressure regulator 66 may include a source of pressurized air. In an exemplary implementation, the air pressure regulator 66 provides pressurized air at about 50 pounds per square inch (psi). Air flow to the nozzle 50 is controlled by the air flow control valve 60 and monitored by the air flow meter 62. Air from the air pressure regulator 66 may be filtered of impurities by the air filter 64.

The chemical flow subassembly 54 may include a chemical flow control valve 70, chemical flow meters 72, and chemical reservoir 74. Flow of a decontaminating fluid 76 from the chemical reservoir 74 to the nozzle 50 may be controlled by the chemical flow control valve 70 and monitored by the chemical flow meters 72. The decontaminating substance may be pushed or pulled from the chemical reservoir 74. The chemical reservoir 74 may be a holding tank or other assembly configured to hold a decontaminating fluid 76. In some embodiments, the decontaminating fluid 76 is a chemical or other substance suitable for use in a sterilization process that complies with the International Organization for Standardization (ISO) standard ISO/TC 198, Sterilization of Healthcare Products and/or the Association for the Advancement of Medical Instrumentation (AAMI) standard ANSI/AAMI/ISO 11140-1:2005, “Sterilization of Healthcare Products—Chemical Indicators—Part I: General Requirements” (Arlington, Va.: AAMI 2005). In some embodiments, the decontaminating fluid 76 is a room temperature (e.g, 20° C. to 25° C.) substance that may be dispersed as an atomized/vaporized solution or fog during the decontamination process. For example, the decontaminating fluid 76 may include H₂O₂, PAA, and/or acetic acid. The decontaminating fluid 76 may also include water. In one exemplary implementation, the decontaminating fluid 76 includes H₂O₂, PAA, and/or acetic acid, and from about 50% to about 75% weight water by weight of the decontaminating fluid 76.

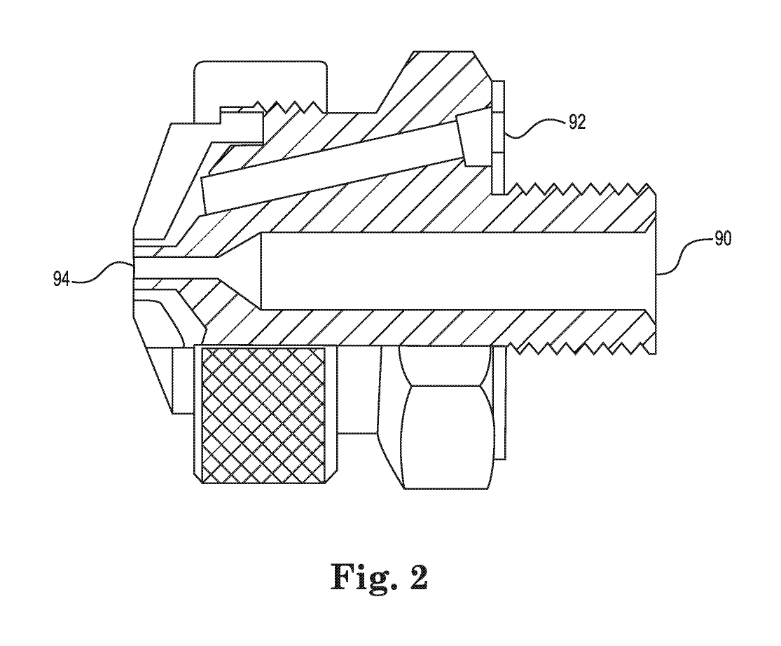

In some embodiments, the nozzle 50 is an atomizing nozzle that is configured to transform the decontaminating fluid 76 at the input of the nozzle 50 to an atomized/vaporized solution at the output of the nozzle 50. FIG. 2 is a cutaway view showing a cross section of one possible embodiment of an atomizing nozzle 50, which includes a gas feed line 90, a liquid feed line 92, and an outlet 94. In use, an inlet gas flows through the gas feed line 90 and a liquid flows through the liquid feed line 92.

The gas feed line 90 may be connected to a supply of pressurized or compressed air that forms the inlet gas. For example, the gas feed line 90 may be connected by a hose to a pump or air compressor that may provide pressurized air to the gas feed line 90 on demand. In another example, the gas feed line 90 may be connected by a hose to a tank that contains compressed air which comprises the inlet gas provided to the atomizing nozzle 50. In one embodiment, the gas feed line 90 may be connected to an air flow subassembly 52 as in FIG. 1, that may include an air flow control valve 60, an air flow meter 62, an air filter 64, and an air pressure regulator 66. In one embodiment, the inlet gas may be heated prior to being supplied to the gas feed line 90. For example, the inlet gas may be heated as it flows through the gas feed line 90.

The liquid feed line 92 may be connected by a hose or tube to a source of decontaminating fluid 76. In one embodiment, the liquid feed line 92 may be connected to a chemical flow subassembly 54 as in FIG. 1. The chemical flow subassembly 54 may include a chemical flow control valve 70, chemical flow meters 72, and chemical reservoir 74. The chemical reservoir 74 may be a holding tank or other assembly configured to hold a decontaminating fluid 76. For example, the source of decontaminating fluid 76 may be a tank containing suitable chemicals mixed together. In another example, the source of decontaminating fluid 76 may be multiple hoses that each connect to a source of an individual chemical component, and the multiple hoses may combine into one hose prior to flowing into the liquid feed line 92. In this example, a user may control the concentration of each chemical in the decontaminating fluid 76 as the decontaminating fluid 76 is being supplied to the liquid feed line 92.

In one embodiment, the gas and the liquid may be mixed by the atomizing nozzle 50 and a gas and liquid mixture may be dispersed at outlet 94. In FIG. 2 the gas feed line 90 is located towards the center of the atomizing nozzle 50 and a liquid feed line 92 is located between the center and the side of the atomizing nozzle 50 when viewed in the coaxial direction. However, the gas feed line 90 and liquid feed line 92 may be positioned at any suitable location within the atomizing nozzle 50. In some embodiments, the liquid feed line 92 may join the gas feed line 90 before the outlet 94. That is, the liquid and the gas may be mixed prior to the outlet 94. Alternatively, the liquid feed line 92 may join the gas feed line 90 at the outlet 94. In one embodiment, gas exiting the gas feed line 90 may be used to drive liquid out of the liquid feed line 92. The atomizing nozzle 50 as shown includes a single gas feed line 90 and a single liquid feed line 92. However, the atomizing nozzle 50 may include two or more gas feed lines 90 and/or liquid feed lines 92. Other possible nozzle designs that may be used include hollow cone, whirl-chamber hollow cone, grooved cone, solid cone, flat spray, deflector jet, impinging jet, two-fluid, bypass, poppet, rotary wheels, and ultrasound atomizers.

The atomizing nozzle 50 is used to disperse the decontaminating fluid 76 throughout the air within the decontamination chamber 14. For example, the atomizing nozzle 50 may be configured to receive decontaminating fluid 76 from a liquid feed line 92 as a liquid column and disperse it throughout the decontamination chamber 14 as droplets or a fog. The atomizing nozzle 50 may be configured to receive decontaminating fluid 76 from a liquid feed line 92 as a liquid column and disperse it throughout the decontamination chamber 14 as a vapor.

One mechanism for converting a liquid column into droplets, fog, or vapor is to use a high velocity flow of air near or around the liquid column to create a turbulence field. In this example, local variations in turbulent pressure distort the liquid column to form droplets. If a velocity field with adequate turbulence is provided, the droplets may break up further to form ever smaller droplets or vapor. In one embodiment, a turbulence field may be provided by gas exiting the gas feed line 90. For example, the gas exiting the gas feed line 90 may be provided at a high enough velocity to create the turbulence field. Another mechanism for converting a liquid column into droplets is by mechanical impingement that breaks up the liquid column as it exits the nozzle.

To produce the atomized/vaporized solution, the atomizing nozzle 50 may generate fine droplets of the decontaminating fluid 76 that average, for example, less than about 10 μm in diameter or width. Droplets of this size tend to bounce off of solid surfaces, allowing for even dispersion, while avoiding excessive condensation, corrosion, and surface wetting issues in the decontamination chamber 14. In addition, the small droplets evaporate, and the vapor penetrates normally inaccessible areas, resulting in a more effective process. In some embodiments, the droplets of decontaminating fluid 76 are 10 μm diameter droplets with an evaporation rate of 50-375 ms at between 0-75% relative humidity (RH). One example nozzle 50 that may be suitable for use in the system 10 is a nozzle such as that used in the Minncare Dry Fog® or Mini Dry Fog systems, sold by Mar Cor Purification, Skippack, Pa. Another example nozzle 50 that may be suitable for use in the system 10 is a spray nozzle assembly including Spraying Systems Co. product numbers 1/4J-316SS, SU1A-316SS, and 46138-16-316SS, sold by Spraying Systems Co., Wheaton, Ill.

In some embodiments, the atomizing nozzle 50 may remain at the temperature of the surroundings throughout the decontamination cycle. For example, the atomizing nozzle 50 may be at ambient or room temperature throughout the decontamination cycle. In some embodiments, the atomizing nozzle 50, may be maintained within a suitable temperature range throughout a decontamination cycle. For example, the atomizing nozzle 50 may operate from about 1° C. to about 100° C., from about 2° C. to about 55° C., from about 3° C. to about 30° C., from about 4° C. to about 20° C., or any temperature within. In some embodiments, the atomizing nozzle 50 may be maintained at 30° C. or below throughout the decontamination cycle. In some embodiments, the atomizing nozzle 50 may be maintained from about 30° C. to about 55° C. throughout a decontamination cycle. For example, in some embodiments, the atomizing nozzle 50 may be maintained from about 4° C. to about 55° C. In some embodiments, the atomizing nozzle 50 may be maintained from about 4° C. to about 18° C. In some embodiments, the atomizing nozzle 50 may be maintained from about 18° C. to about 55° C. throughout a decontamination cycle. In some embodiments, the atomizing nozzle 50 may be maintained from about 30° C. to about 55° C. throughout a decontamination cycle.

In some embodiments, the decontaminating fluid 76 may remain at the temperature of the surroundings throughout the decontamination cycle. For example, the decontaminating fluid 76 may be at ambient or room temperature throughout the decontamination cycle. In some embodiments, the decontaminating fluid 76, may be maintained within a suitable temperature range throughout a decontamination cycle. For example, the decontaminating fluid 76 may be from about 1° C. to about 100° C., from about 2° C. to about 55° C., from about 3° C. to about 30° C., from about 4° C. to about 20° C., or any temperature in between. In some embodiments, the decontaminating fluid 76 may be maintained at 30° C. or below throughout the decontamination cycle. In some embodiments, the decontaminating fluid 76 may be maintained from about 30° C. to about 55° C. throughout a decontamination cycle. In some embodiments, the decontaminating fluid 76 may be maintained at 30° C. or below throughout the decontamination cycle. For example, in some embodiments, the decontaminating fluid 76 may be maintained from about 4° C. to about 55° C. throughout a decontamination cycle. In some embodiments, the decontaminating fluid 76 may be maintained from about 4° C. to about 18° C. In some embodiments, the decontaminating fluid 76 may be maintained from about 18° C. to about 55° C. throughout a decontamination cycle. In some embodiments, the decontaminating fluid 76 may be maintained from about 30° C. to about 55° C. throughout a decontamination cycle.

The amount of atomized/vaporized solution generated by the chemical dispersion assembly 18 may be controlled by the system controller 12. For example, the system controller may control the rate and/or amount of the decontaminating fluid 76 that flows through the nozzle 50. The rate and amount of decontaminating fluid 76 that flows through the nozzle 50 may be preprogrammed into the system controller 12 or may be manually entered into the system controller 12 by a user of the system 10. In addition, the system controller 12 may include multiple programs that provide different rates and amounts of the decontaminating fluid 76 to the nozzle 50.

In some embodiments, the relative humidity, the temperature within the decontamination chamber 14, the temperature of the atomizing nozzle 50, and the temperature of the decontaminating fluid 76, may all be controlled independently. For example, the temperature of the decontamination chamber 14, the temperature of the atomizing nozzle 50, and the temperature of the decontaminating fluid 76 may be maintained at the same temperature or may be controlled to be at different temperatures from each other.

The temperature of the decontamination chamber 14, the temperature of the atomizing nozzle 50, and the temperature of the decontaminating fluid 76 may also be controlled to be at a particular temperature at various levels of relative humidity. For example, the temperature of any of the decontamination chamber 14, the temperature of the atomizing nozzle 50, and the temperature of the decontaminating fluid 76 may be from about 1° C. to about 100° C., from about 2° C. to about 55° C., from about 3° C. to about 30° C., from about 4° C. to about 20° C., or any temperature in between, while the relative humidity within the decontamination chamber 14 may be from about 1% to about 90%, from about 5% to about 75%, from about 10% to about 50%, from about 15% to about 45%, or any value in between. In some embodiments, the temperature of any of the decontamination chamber 14, the temperature of the atomizing nozzle 50, and the temperature of the decontaminating fluid 76 may be from about 1° C. to about 100° C., from about 10° C. to about 80° C., from about 20° C. to about 65° C., from about 30° C. to about 55° C., or any temperature in between, while the relative humidity within the decontamination chamber 14 may be from about 1% to about 90%, from about 5% to about 75%, from about 10% to about 50%, from about 15% to about 45%, or any value in between.

In some embodiments, an atomizer purge valve 80 is connected between the air flow subassembly 52 and chemical flow subassembly 54. The atomizer purge valve 80 may provide a fluid connection between the air pressure regulator 66 and the chemical flow subassembly 54 input to the nozzle 50. To purge decontaminating substance from the nozzle 50, the air flow control valve 60 and chemical flow control valve 70 are closed and the atomizer purge valve 80 is opened to allow pressurized air from the air pressure regulator 66 to be provided through the chemical input of the nozzle 50, thereby forcing residual decontaminating substance out of the nozzle 50.

In some embodiments, the vent valve 20 may allow air to be drawn into the decontamination chamber 14 during venting steps of the decontamination cycle. For example, if the pressure in the decontamination chamber 14 is below atmospheric pressure, the vent valve 20 may be opened to raise the pressure in the decontamination chamber 14. An air filter 84 may be disposed along the intake to the vent valve 20 to remove contaminants from the air stream flowing into the decontamination chamber 14 during venting.

As discussed above, the system controller 12 may operate the components of the system 10 to decontaminate a device or article in the decontamination chamber 14. The system controller 12 may include one or more selectable preprogrammed decontamination cycles that are a function of the device characteristics and desired level of decontamination. Alternatively, cycle parameters may be input into the system controller 12 by a user. As a part of the decontamination cycle, the system controller 12 may monitor and control the environment within the decontamination chamber 14 to improve the efficacy of the decontamination process. For example, the temperature, relative humidity, and pressure within the decontamination chamber 14 may be monitored via the temperature sensor 22, relative humidity sensor 24, and pressure sensor 31, respectively, that are operatively connected to the decontamination chamber 14. The system 10 may include a temperature control element (not shown) associated with the decontamination chamber 14 to control the temperature in the decontamination chamber 14. The pressure within the decontamination chamber 14 is controllable with the vacuum pumps 30, air pressure regulator 66, and vent valve 20.

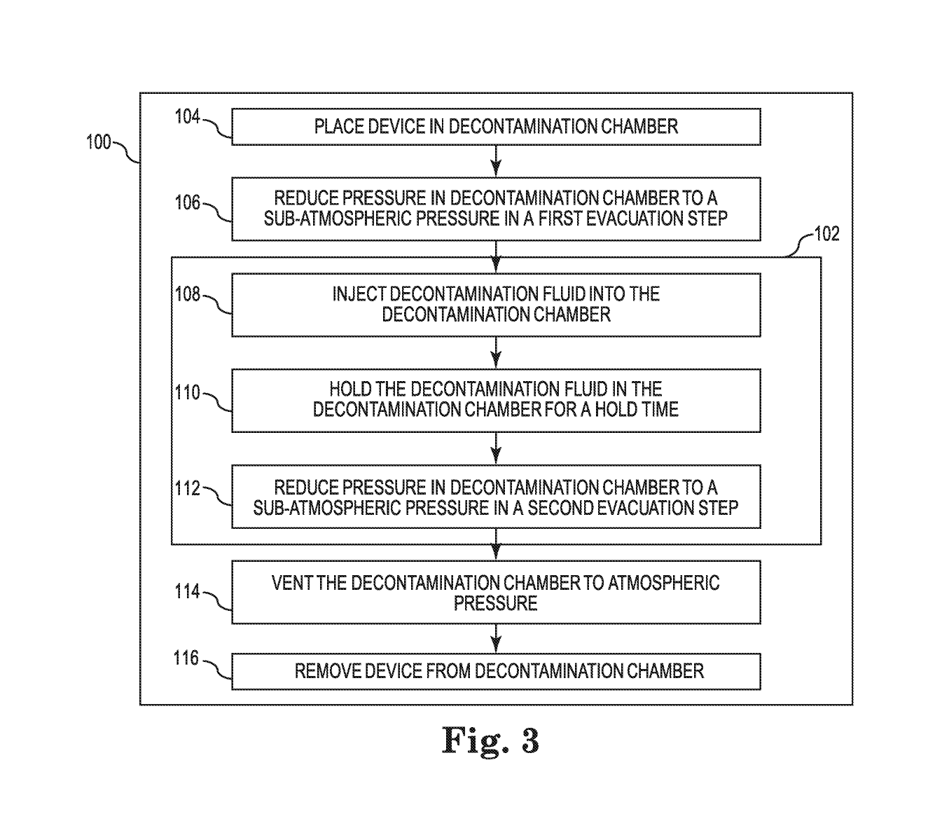

FIG. 3 is a flow diagram of an embodiment of a process for decontaminating a device in the system 10 of FIG. 1. In one embodiment, a decontamination cycle 100 begins when a device to be decontaminated is placed within the decontamination chamber 14 and ends when the device is removed from the decontamination chamber 14. In step 104, a device to be decontaminated is placed inside the decontamination chamber 14, and the door D is closed to seal the interior of the decontamination chamber 14.

After placing the device within the decontamination chamber 14 and prior to injecting a decontaminating fluid 76 into the decontamination chamber 14, the device to be decontaminated may be preconditioned. For example, the device may be allowed to reach ambient temperature, less than about 30° C., and more specifically between about 18° C. and about 30° C. The preconditioning of the device to ambient temperature may improve the efficacy of the decontamination process of the system 10. Alternatively, the device may be preconditioned to ambient temperature before placement into the decontamination chamber 14.

The atmosphere within the decontamination chamber 14 may also be preconditioned after the device is placed within the decontamination chamber 14. For example, that atmosphere may be preconditioned to a relative humidity equivalent to that of the ambient relative humidity. Alternatively, the atmosphere may be preconditioned to a specified relative humidity. In some embodiments, the relative humidity in the decontamination chamber 14 is preconditioned to less than about 50% relative humidity.

In some embodiments, a pressure within the decontamination chamber 14 may be reduced after a device to be decontaminated has been placed inside. For example, in step 106, the pressure in the decontamination chamber 14 is reduced to within a sub-atmospheric pressure range in a first evacuation step 106. In some embodiments a sub-atmospheric pressure range may be a pressure lower than the pressure outside of the decontamination chamber 14. For example, the pressure in the decontamination chamber 14 may be reduced to within a sub-atmospheric pressure range in a first evacuation step 106. For example, the pressure control assembly 16 may evacuate the decontamination chamber 14 to produce a vacuum or partial vacuum in the decontamination chamber 14 relative to the air pressure outside the decontamination chamber 14. In some embodiments, a sub-atmospheric pressure may be a pressure below a particular measured threshold. In some embodiments, the pressure in the decontamination chamber 14 is reduced to less than 30 torr, and in some embodiments to less than 10 torr. For example, in some embodiments, the pressure in the decontamination chamber 14 may be reduced to from about 0.0005 torr to about 30 torr, from about 0.0025 torr to about 20 torr or from about 0.005 torr to about 10 torr.

In some embodiments, after the pressure in the decontamination chamber 14 reaches the programmed sub-atmospheric pressure, the chemical injection cycle 102 may begin by injecting the decontaminating fluid 76 into the decontamination chamber 14. The chemical injection cycle 102 may continue until the decontamination chamber 14 is evacuated as in step 112. In some embodiments, during the chemical injection cycle 102 a dispersion assembly 18 may add decontaminating fluid 76 into the decontamination chamber 14 at step 108. For example, the chemical dispersion assembly 18 may disperse or inject the decontaminating fluid 76 into the decontamination chamber 14 at a first rate. According to various embodiments, the first rate is selected such that the water content of the decontaminating fluid 76 increases the relative humidity and/or pressure in the decontamination chamber 14. In some embodiments, between 0.5 ml and not greater than 20 ml of the decontaminating fluid 76 is added to the decontamination chamber 14.

In some embodiments, the decontaminating fluid 76 is dispersed at the first rate until the relative humidity is in the range of more than 0% and not more than 50%. For example, in some embodiments decontaminating fluid 76 is dispersed until the relative humidity is in the range of more than 0% to not greater than 45%. In some embodiments, the relative humidity is not greater than 45%, and more particularly, not greater than 20% following dispersion of the decontaminating fluid 76 at a first rate. In some embodiments, decontaminating fluid 76 is added until the concentration of decontaminating fluid 76 per decontamination chamber 14 volume is between 1 and 75 mg/L.

In some embodiments, while dispersing the decontaminating fluid 76, the pressure in the decontamination chamber 14 may increase. The pressure increase may be effected by the air pressure regulator 66 of the chemical dispersion assembly 18. That is, the air pressure that is employed to disperse the decontaminating fluid 76 from the nozzle 50 may be used to increase the pressure in the decontamination chamber 14. Alternatively, the pressure increase may be produced, at least in part, by opening the vent valve 20.

In some embodiments, after the addition of the decontaminating fluid 76 at the first rate, the decontaminating fluid 76 may be added into the decontamination chamber 14 at a second rate. In some embodiments, the second rate is higher than the first rate of step 108. In some embodiments, the duration of adding at the second rate is set such that the concentration or density of the decontaminating fluid 76 reaches predetermined levels inside the decontamination chamber 14 at the end of dispersion at the second rate. The predetermined concentration of the decontaminating fluid 76 may be set in the decontamination cycle selected or programmed by the user on the system controller 12, and may be a function of the preferred or required level of decontamination of the device. The introduction of the decontaminating fluid 76 through the nozzle 50 with air flow subassembly 52 provides good circulation and coverage of the decontaminating fluid 76 within the decontamination chamber 14.

In some embodiments, if a decontaminating fluid 76 is dispersed at a second rate, the pressure inside the decontamination chamber 14 may be increased. Again, a pressure increase may be effected by the air pressure regulator 66 of the chemical dispersion assembly 18. Alternatively, a pressure increase may be produced, at least in part, by opening the vent valve 20. After adding the decontaminating fluid 76 to produce the programmed level of concentration, the pressure inside the decontamination chamber 14 is within a hold pressure range. In some embodiments, the hold pressure is between about 0 and 760 torr, between about 0.0007 and 760 torr, or more particularly between about 0.005 torr and 760 torr. In a preferred embodiment, the hold pressure is between about 150 and about 500 torr.

In step 110, when the decontamination chamber 14 is within the hold pressure range, the conditions inside the decontamination chamber 14 are maintained for a hold time. The decontaminating fluid 76 is held in the decontamination chamber 14 for an amount of time sufficient to decontaminate the medical device disposed therein. This amount of time may be programmed into the system controller 12, and may be based on the size and type of medical device being decontaminated, as well as the content and concentration of the decontaminating fluid 76, or the type of packaging used to contain the device if applicable.

When the decontaminating fluid 76 has been held in the decontamination chamber 14 for the desired or programmed amount of time, in step 110, the system controller 12 may command the pressure control assembly 16 to again evacuate the decontamination chamber 14 to reduce the pressure in the decontamination chamber 14 to within a sub-atmospheric pressure range in a second evacuation step 112. The reduction in pressure draws the environment in the decontamination chamber 14 through the filters 34, 36, 38 and removes the decontaminating fluid 76 from the decontamination chamber 14. In some embodiments, the pressure in the decontamination chamber 14 is reduced to less than 10 torr. In some embodiments, the decontamination chamber 14 is maintained at the reduced pressure of step 112 for a programmed period of time.

The chemical injection cycle 102 steps of adding the decontaminating fluid 76 into the decontamination chamber 14 and/or evacuating the decontamination chamber 14 may be performed a plurality of times. For example, after the decontaminating fluid 76 is evacuated from the decontamination chamber 14 in a first cycle as described above, the decontaminating fluid 76 may be added into the decontamination chamber again in a second chemical injection cycle 102. The decontaminating fluid 76 from the second chemical injection cycle 102 may then be held in the decontamination chamber 14 for a hold time (similar to step 110 described above). The decontamination chamber 14 may subsequently be evacuated to remove the decontaminating fluid 76, as described above in step 112. The system controller 12 may be programmed to repeat the chemical injection cycle 102 any number of times.

In addition, after any iteration of the second evacuation step 112, the system 10 may be programmed to increase the pressure in the decontamination chamber 14 without adding decontaminating fluid 76 into the chamber. For example, in some embodiments, the chemical flow control valve 70 is closed and the air flow control valve 60 is opened to force air into the decontamination chamber 14 to increase pressure. As another example, the vent valve 20 is opened to raise the pressure inside the decontamination chamber 14. After the pressure in the decontamination chamber 14 reaches a desired level (e.g., atmospheric pressure), the decontamination chamber 14 may again be evacuated to a sub-atmospheric pressure. The additional steps of increasing pressure in the decontamination chamber 14 followed by evacuation may be employed to assure complete removal of the decontaminating fluid 76 from the decontamination chamber 14.

In some embodiments, the relative humidity within the decontamination chamber 14 is maintained at or below 45% and in some embodiments at or below 20% during the chemical injection cycle(s) 102. In some embodiments, the relative humidity is from greater than 0% to about 20% following the chemical injection cycle(s) 102, and in some embodiments is from about 10% to about 20% following the chemical injection cycle(s) 102. In some embodiments, the relative humidity in the decontamination chamber 14 is from about 20% to about 50% following the chemical injection cycle(s) 102, and in some embodiments is from about 20% to about 45% following the chemical injection cycle(s) 102.

After the programmed number of cycles or evacuation steps in chemical injection cycle 102, the system controller 12 may open the vent valve 20 to vent the decontamination chamber 14 to atmospheric pressure in step 114. This draws air exterior to the decontamination chamber 14 through the air filter 84 and into the decontamination chamber 14. When the pressure in the decontamination chamber 14 equalizes with the pressure exterior of the decontamination chamber 14, the decontamination chamber 14 may be opened to remove the decontaminated device from the system 10 as in step 116.

In some embodiments, the relative humidity within the decontamination chamber 14 is maintained at or below 45% and in some embodiments at or below 20% throughout the decontamination cycle 100. That is in some embodiments, the relative humidity from a time that the device is place in the decontamination chamber 14 to when the device is removed from the decontamination chamber 14 is maintained at or below 45% and in some embodiments at or below 20%. In some embodiments, the relative humidity in the decontamination chamber 14 is from greater than 0% to about 20% throughout the decontamination cycle 100. In some embodiments, the relative humidity in the decontamination chamber 14 is from about 20% to about 50% throughout the decontamination cycle 100, and in some embodiments is from about 20% to about 45% throughout the decontamination cycle 100. In some embodiments, decontaminating fluid 76 is added until the amount of decontaminating fluid 76 per decontamination chamber 14 volume is between 1 and 75 mg/L.

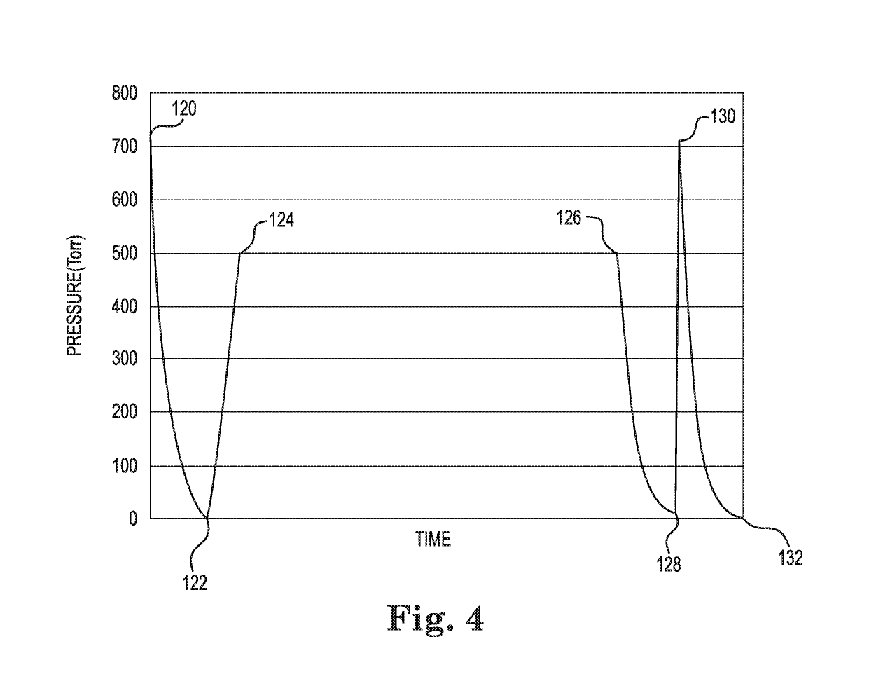

FIG. 4 is a graph showing pressure versus time in an example decontamination cycle. The graph in FIG. 4 illustrates the pressure conditions of the cycle after the device and decontamination chamber 14 have been preconditioned as described above. At time 120, the decontamination chamber 14 is evacuated to a sub-atmospheric pressure in a first evacuation step. In the embodiment shown, the sub-atmospheric pressure is approximately 10 torr. When the programmed sub-atmospheric pressure is reached in the decontamination chamber 14, at time 122, adding the decontaminating fluid 76 as described above in step 108 occurs. After adding the decontaminating fluid 76 as described in step 108, the decontamination chamber 14 is at the hold pressure at time 124. In the embodiment shown, the hold pressure is approximately 500 torr. The conditions in the decontamination chamber 14 are held constant for a hold time until time 126. In some embodiments, the hold time between times 124 and 126 is in the range of approximately five minutes to five hours. After the hold time, the decontamination chamber 14 is evacuated to a sub-atmospheric pressure in a second evacuation step. In the embodiment shown, the sub-atmospheric pressure is approximately 10 torr. When the programmed sub-atmospheric pressure is reached in the decontamination chamber 14, at time 128, the pressure is raised in the decontamination chamber 14. In some embodiments, the pressure in the decontamination chamber 14 is raised to approximately atmospheric pressure. After the programmed pressure is reached in the decontamination chamber 14, at time 130 the decontamination chamber 14 may optionally be evacuated to a sub-atmospheric pressure (e.g., 10 torr), and subsequently vented to atmospheric pressure. The additional evacuation step starting at time 130 is optional, and may be employed to assure complete removal of the decontaminating fluid 76 from the decontamination chamber 14.

To ensure adequate decontamination, the decontaminated devices may be tested for microbial activity following a decontamination cycle. As described herein, a decontamination cycle includes at least one release of decontaminating substance into the decontamination chamber 14. A sterility assurance level (SAL) is one common measurement used to describe the sterility or decontamination achieved by a system or process. A SAL is the probability of a device being non-sterile after it has been subjected to a sterilization process, the probability of which is a very low number and thus is expressed as a negative exponent. For example, in some embodiments, the SAL of the decontamination cycle described herein may be less than 10 to the minus six (10⁻⁶). That is, in some embodiments, the SAL of the described cycle may achieve a three-log reduction in microbial population. In some embodiments, a lower overall SAL may be achieved by repeating a decontamination cycle multiple times. For example, a decontamination cycle that provides a SAL of 10 to the minus three (10⁻³) may be repeated two times to provide an overall decontamination process SAL of 10 to the minus six (10⁻⁶).

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

Ten BIs containing Geobacillus stearothermophilus (ATCC® 7593™ and ATCC® 12980™ available from American Type Culture Collection of Manassas, Va.) were placed within a decontamination chamber at room temperature and atmospheric pressure. The pressure inside the chamber was reduced to about 10 torr. Two and a half mL of decontaminating fluid containing PAA at 5% concentration was added to the decontamination chamber. After holding the BIs in the chamber for 30 seconds at these conditions, the chamber was returned to atmospheric pressure. The BIs were removed from the chamber. Testing showed that five out of the ten BIs were killed. This trial produced a 6 log reduction of Geobacillus in 0.48 minutes (28.8 seconds), thus the kill rate was 0.08 minutes per log of the most resistant organism (MRO). The D-value for this example was determined to be 0.08.

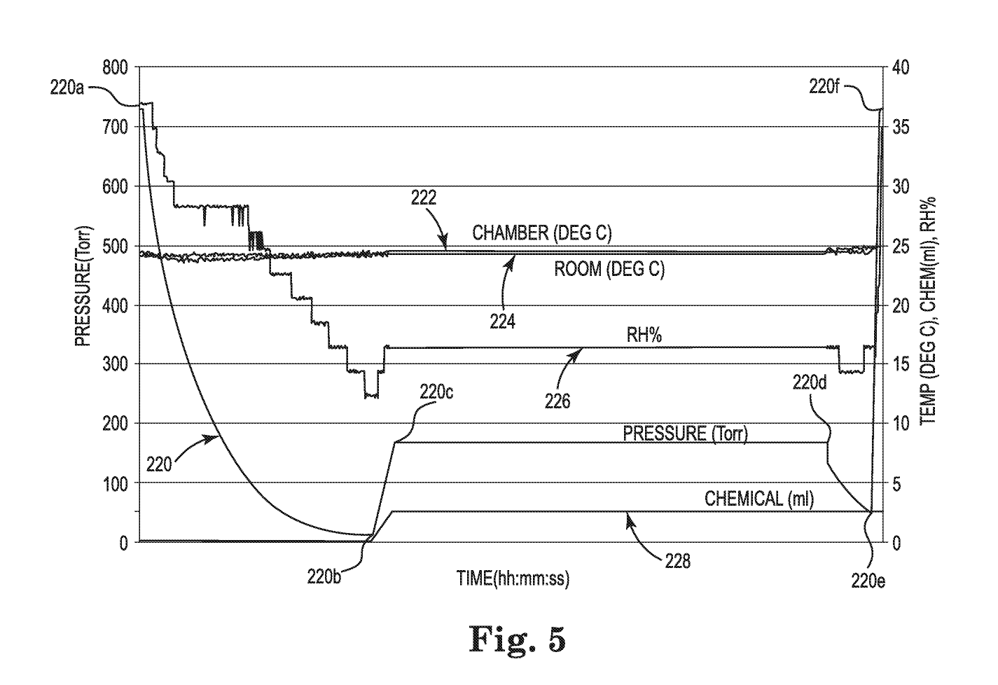

FIG. 5 is a graph showing the chamber pressure in torr (line 220), the chamber temperature in ° C. (line 222), the room temperature in ° C. (line 224), the relative humidity in % (line 226), and the amount of decontaminating fluid added in mL (line 228). The beginning of a decontamination cycle is shown at the point 220a. At time 220a the pressure within the decontamination chamber was reduced, which can be seen by following line 220, until it reached a suitable pressure, which is shown on the graph at 220b. As the pressure inside the chamber was reduced, the relative humidity inside the chamber also decreased, which can be seen by following line 226 which shows the relative humidity. At the time designated 220b, the pressure within the decontamination chamber was about 10 torr and the relative humidity was about 12%. At this time, 2.5 mL of decontaminating fluid was dispersed into the decontamination chamber. Line 228 illustrates the amount of decontaminating fluid in the chamber increasing at a time pointed out as 220b. Dispersing decontaminating into the chamber increased the pressure and relative humidity inside the chamber, which can be seen by following line 220 between points 220b and 220c, and line 226 for the corresponding time interval.

The pressure stopped increasing once no more decontaminating fluid was added into the chamber. After the decontaminating fluid was added to the chamber, the relative humidity was about 16.5% and the pressure was about 160 torr. This time is shown on the graph as 220c. The pressure and relative humidity in the chamber were then held constant for a hold time, shown as the time between points 220c and 220d on the graph. The time interval in FIG. 5 showing the hold time from 220c to 220d has been expanded for illustrative purposes and is not meant to indicate the actual time duration in proportion to the time intervals of the other steps. At 220d, the end of the hold time, the pressure inside the chamber was reduced until it reached 50 torr, as shown on the graphs as 220e. The relative humidity in the chamber at the time corresponding to point 220e was about 14%. As can been seen from following line 220 between points 220e and 220f, the pressure in the chamber was then increased to atmospheric pressure.

By following line 222 which shows the chamber temperature and line 224 which shows the room temperature, it can be seen that the temperature of the chamber remained below 30° C. throughout the decontamination cycle. Also, the decontaminating fluid was controlled such that the decontaminating fluid never exceeded 55° C. for longer than one second. It is believed that using these temperatures and pressures, no formaldehyde was formed during the decontamination process.

### Example 2

Similar to Example 1, ten BIs containing Geobacillus stearothermophilus (ATCC® 7593™ and ATCC® 12980™ available from American Type Culture Collection of Manassas, Va.) were placed within a decontamination chamber at room temperature and atmospheric pressure. The pressure inside the chamber was reduced to about 10 torr. Two and a half mL of decontaminating fluid containing PAA at 5% concentration was added to the decontamination chamber. However, in this example, the hold time was extended to about three minutes. Surprisingly, although the relative humidity in the chamber never exceeded 20%, using a three minute hold time resulted in all ten BIs being killed, or a 100% kill rate. The process parameters and procedure used were the same as in Example 1. Because all of the BIs were killed, the D-value using this three minute cycle could not be calculated.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

The following is claimed:

1. A decontamination apparatus for decontaminating a device, the decontamination apparatus comprising:
a decontamination chamber;
an atomizing nozzle in fluid communication with a decontamination fluid containing water and peracetic acid, the atomizing nozzle configured to supply the decontamination fluid to the decontamination chamber as a vapor; and
a control system configured to control a flow rate of the decontamination fluid into the decontamination chamber such that the relative humidity within the decontamination chamber is maintained between 0% and 45% throughout a complete chemical injection cycle, and wherein the control system is configured to control a decontamination cycle that includes the chemical injection cycle to achieve a sterility assurance level of at least 10 to the minus three.

2. The decontamination apparatus of claim 1, wherein the decontamination cycle is defined from when a device is placed in the decontamination chamber to when the device is removed from the decontamination chamber.

3. The decontamination apparatus of claim 1, wherein the control system maintains the relative humidity within the decontamination chamber from about 20% to about 45% throughout the decontamination cycle.

4. The decontamination apparatus of claim 1, wherein the control system maintains the relative humidity within the decontamination chamber below 20% throughout the chemical injection cycle.

5. The decontamination apparatus of claim 1, wherein the decontamination chamber is maintained below 30° C. throughout the decontamination cycle.

6. The decontamination apparatus of claim 1, wherein the decontamination chamber is maintained from about 30° C. to about 55° C. throughout the decontamination cycle.

7. The decontamination apparatus of claim 1, further comprising a sensor for measuring a relative humidity within the decontamination chamber.

8. The decontamination apparatus of claim 1, wherein the system controller is configured to control the decontamination chamber, chemical dispersion assembly, and pressure control assembly to:
reduce a pressure in the decontamination chamber to within a sub-atmospheric pressure range in a first evacuation step prior to the addition of the decontamination fluid to the decontamination chamber;
remove the decontamination fluid from the decontamination chamber in a second evacuation step; and
vent the decontamination chamber to atmospheric pressure after removal of the decontamination fluid from the decontamination chamber.

9. The decontamination apparatus of claim 1, wherein controlling the flow rate of the decontamination fluid into the decontamination chamber includes controlling a first flow rate of the decontamination fluid into the decontamination chamber and controlling a second flow rate of the decontamination fluid into the decontamination chamber which is different than the first flow rate, wherein the relative humidity within the decontamination chamber is maintained between 0% and 45% during the addition of the decontamination fluid at the first flow rate and the second flow rate.

10. The decontamination apparatus of claim 1, wherein the control system is configured to maintain the relative humidity inside the decontamination chamber at or below 17% throughout a complete chemical injection cycle.

11. The decontamination apparatus of claim 1, wherein the control system is configured to maintain the relative humidity inside the decontamination chamber at or below 17% throughout a complete chemical injection cycle and expose the device to decontaminating fluid for no longer than three minutes.

12. A method of decontaminating a device, the method comprising:
dispersing a decontamination fluid as a vapor into a decontamination chamber containing a device to be decontaminated, the decontamination fluid containing peracetic acid and water;
controlling a dispersion rate of the decontamination fluid such that the environment within the decontamination chamber is below 45% relative humidity throughout a decontamination cycle defined from when the device is placed in the decontamination chamber to when the device is removed from the decontamination chamber, wherein the decontamination cycle achieves a sterility assurance level of at least 10 to the minus three.

13. The method of claim 12, wherein the dispersion rate of the decontamination fluid is controlled such that the environment within the chamber is between 20% and 45% relative humidity throughout the decontamination cycle.

14. The method of claim 12, wherein the dispersion rate of the decontamination fluid is controlled such that the environment is below 20% relative humidity after the decontamination fluid is dispersed into the decontamination chamber.

15. The method of claim 12, wherein the dispersion rate of the decontamination fluid is controlled such that the environment is 17% relative humidity or lower after the decontamination fluid is dispersed into the decontamination chamber.

16. The method of claim 12, wherein the dispersion rate of the decontamination fluid is controlled such that the environment is 17% relative humidity or lower after the decontamination fluid is dispersed into the decontamination chamber and the device to be decontaminated is exposed to the decontamination fluid for less than three minutes.

17. The method of claim 12, wherein prior to dispersing the decontamination fluid into the decontamination chamber, the relative humidity of the decontamination chamber is below 45%.

18. The method of claim 12, wherein prior to dispersing the decontamination fluid into the decontamination chamber, a pressure in the decontamination chamber is sub-atmospheric.

19. The method of claim 12, wherein a temperature in the decontamination chamber is below 30° C. throughout the decontamination cycle.

20. The method of claim 12, wherein a temperature in the decontamination chamber is from about 30° C. to about 55° C. throughout the decontamination cycle.
